(19) United States
(12) Patent Application Publication
(10) Pub. No.: US 2022/0313981 A1
(43) Pub. Date: Oct. 6, 2022

(54) PHARYNGEAL ELECTRICAL STIMULATION DEVICES, SYSTEMS, AND METHODS

(71) Applicant: Phagenesis Limited, Manchester (GB)

(72) Inventor: Conor Mulrooney, Manchester (GB)

(21) Appl. No.: 17/658,035

(22) Filed: Apr. 5, 2022

Related U.S. Application Data
(60) Provisional application No. 63/200,977, filed on Apr. 6, 2021.

(51) Int. Cl. A61N 1/04; A61N 1/05; A61N 1/36

(52) U.S. Cl. CPC A61N 1/0452; A61N 1/0517; A61N 1/0519; A61N 1/3603; A61N 1/36003

(57) ABSTRACT

Electrical stimulation devices, systems, and methods are disclosed herein. Various embodiments of the present technology, for example, are directed to a method of identifying a treatment location for applying electrical stimulation energy to treat a medical condition. A method in accordance with some embodiments of the present technology comprises identifying a treatment location in a pharynx of the patient such that application of electrical stimulation energy at the treatment location produces a favorable increase in motor activity and/or functional reorganization of the centers in the brain responsible for controlling and coordinating motor activity of the pharynx.

### CROSS-REFERENCE TO RELATED APPLICATION(S)

The present application claims the benefit of priority to U.S. Application No. 63/200,977, filed Apr. 6, 2021, which is incorporated by reference herein in its entirety.

### TECHNICAL FIELD

The present technology relates to pharyngeal electrical stimulation devices, systems, and methods. Various embodiments of the present technology relate to devices, systems, and methods for identifying a preferred treatment location for the application of electrical stimulation energy to treat a medical condition.

### BACKGROUND

Dysphagia is the condition whereby a patient has difficulty in swallowing or is unable to swallow safely. Dysphagia may be caused, for example, by stroke, neurodegenerative diseases, brain tumors, or in some cases by other co-morbidities, such as respiratory disorders. It has been reported that between 7 and 10% of all adults older than 50 years of age present with clinically significant dysphagia. Of those over the age of 60, this increases to 14%. In total, 10 million Americans are evaluated each year in clinics and hospitals for swallowing difficulties. It has also been reported that over 51% of institutionalized elderly patients present with oropharyngeal dysphagia. Complications that have been associated with dysphagia include pneumonia, malnutrition, dehydration, poorer long-term outcome, increased length of hospital stay, increased rehabilitation time and the need for long-term care assistance, increased mortality, and increased health care costs. These complications impact the physical and social wellbeing of patients, quality of life of both patients and caregivers, and the utilization of health care resources.

Swallowing is a complex sensorimotor activity, which depends on highly organized interactions among the cerebral cortex, the brainstem, and the peripheral nervous system. This process has both voluntary and involuntary components, reflecting central regulatory pathways within swallowing centers in the cortex and brainstem respectively. Sensory feedback is also critical to swallowing. Stimulation of afferent fibers from cranial nerves V, IX and X can initiate or modulate the reflex swallowing process in animals, whereas a reduction in oropharyngeal sensation, by local anesthesia or afferent nerve damage, can disrupt the normal pattern of volitionally initiated swallowing.

In view of the above, there remains a need for improved devices, systems, and methods that can treat dysphagia.

### SUMMARY

The present technology relates to pharyngeal electrical stimulation devices and associated systems and methods. In particular embodiments, the present technology comprises a method of identifying a preferred treatment location for the application of electrical stimulation energy to treat one or more medical conditions, such as dysphagia. A method in accordance with some embodiments of the present technology comprises identifying a treatment location in a pharynx of the patient at which the application of electrical stimulation energy produces an increase in motor activity and/or functional reorganization of the centers in the brain responsible for controlling and coordinating the motor activity. For example, delivering electrical stimulation energy to nerves proximate the pharynx can cause functional reorganization of the swallow motor cortex and/or an increase in cortical excitability that improves swallowing function and treats dysphagia. The subject technology is illustrated, for example, according to various aspects described below, including with reference to FIGS. 1-5. Various examples of aspects of the subject technology are described as numbered clauses (1, 2, 3, etc.) for convenience. These are provided as examples and do not limit the subject technology.

1. A method comprising:
via a nasal or oral cavity, positioning an elongate member carrying a conductive element in a lumen of a pharynx of a human subject such that the conductive element is positioned at a first location;
applying electrical stimulation energy at the first location via the conductive element;
after applying the electrical stimulation energy at the first location, obtaining first data characterizing a cortical excitability of the subject;
positioning the elongate member in the lumen such that the conductive element is positioned at a second location;
applying electrical stimulation energy at the second location via the conductive element;
after applying the electrical stimulation energy at the second location, obtaining second data characterizing the cortical excitability of the subject; and
comparing the cortical excitability of the first data to the cortical excitability of the second data.

2. The method of Clause 1, further comprising, based on the comparison, determining whether the application of electrical stimulation energy at the first location or the second location results in greater cortical excitability.

3. The method of Clause 1 or Clause 2, wherein obtaining first data characterizing a cortical excitability includes:
stimulating the subject's neural tissue via transcranial magnetic stimulation (TMS), and
detecting a pharyngeal electromyographic (EMG) response to the stimulation.

4. The method of any one of Clauses 1 to 3, wherein obtaining the second data characterizing the cortical excitability includes:
stimulating the subject's neural tissue via TMS, and
detecting a pharyngeal EMG response to the stimulation.

5. The method of Clause 3 or Clause 4, wherein stimulating the subject's neural tissue comprises stimulating one or more regions of the subject's swallow motor cortex.

6. The method of any one of Clauses 3 to 5, wherein stimulating the subject's neural tissue comprises stimulating one or more of the subject's caudal sensory motor cortex, anterior insula, premotor cortex, frontal operculum, anterior cingulate cortex, prefrontal cortex, anterolateral parietal cortex, posterior parietal cortex, precuneus, superiomedial temporal cortex, posterior cingulate cortex, putamen, or caudal nuclei.

7. The method of any one of Clauses 3 to 6, wherein detecting the pharyngeal EMG response includes obtaining a motor-evoked potential (MEP) parameter.

8. The method of Clause 7, wherein the MEP parameter includes an MEP amplitude and/or an MEP latency.

9. The method of any one of Clauses 1 to 8, further comprising:
obtaining first baseline data characterizing a cortical excitability of the subject before applying the electrical stimulation energy at the first location;
obtaining second baseline data characterizing a cortical excitability of the subject before applying the electrical stimulation energy at the second location;
comparing the cortical excitability associated with the first data to the cortical excitability associated with the first baseline data; and
comparing the cortical excitability associated with the second data to the cortical excitability of the second baseline data.

10. A method comprising:
obtaining baseline data characterizing a swallowing function parameter of a subject;
delivering electrical stimulation energy at a first location within a pharynx of the subject;
during and/or after delivering energy at the first location, obtaining first data characterizing the swallowing function parameter;
delivering electrical stimulation energy at a second location within the pharynx;
during and/or after delivering energy at the second location, obtaining second data characterizing the swallowing function parameter; and
based on at least two of the baseline data, the first data, or the second data, identifying which of the first or second locations is a preferred treatment location for delivering electrical stimulation energy.

11. The method of Clause 10, wherein the swallowing function parameter is a cortical representation associated with the pharynx.

12. The method of Clause 10 or Clause 11, wherein the swallowing function parameter is a cortical excitability.

13. The method of any one of Clauses 10 to 12, wherein the swallowing function parameter is an outcome measure.

14. The method of any one of Clauses 10 to 13, wherein obtaining the baseline data comprises obtaining first baseline data, the method further comprising, after delivering electrical stimulation energy at the first location and before delivering electrical stimulation energy at the second location, obtaining second baseline data characterizing the swallowing function parameter.

15. The method of any one of Clauses 10 to 14, wherein obtaining the baseline data, the first data, and/or the second data comprises:
applying magnetic stimulation energy to one or more regions of the patient's neural tissue; and
during and/or after applying the magnetic stimulation energy to each of the one or more regions, detecting a pharyngeal EMG response to the magnetic stimulation energy.

16. The method of any one of Clauses 10 to 15, wherein obtaining the baseline data, the first data, and/or the second data comprises performing functional magnetic resonance imaging of the subject's brain.

17. The method of any one of Clauses 10 to 16, wherein identifying which of the first or second locations is the preferred treatment location comprises:
determining a first difference between the baseline data and the first data;
determining a second difference between the baseline data and the second data; and
comparing the first and second differences.

18. The method of Clause 17, wherein identifying which of the first or second locations is the preferred treatment location comprises identifying which of the first and second differences is greater.

19. The method of any one of Clauses 13 to 18, wherein identifying which of the first or second locations is the preferred treatment location comprises identifying a percentage of the one or more regions of the patient's neural tissue that, when stimulated, produce a pharyngeal EMG response above a predetermined threshold for the first data and the second data

20. The method of Clause 19, wherein identifying which of the first or second locations is the preferred treatment location comprises identifying which of the first or second data has a greater percentage of the one or more regions of the patient's neural tissue that, when stimulated, produce a pharyngeal EMG response above the predetermined threshold.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a fragmentary sagittal view of a pharynx of a human patient.

FIG. 2 depicts an electrical stimulation system in accordance with several embodiments of the present technology.

FIGS. 3A and 3B depict an electrical stimulation device transnasally inserted into a pharynx of a patient in accordance with several embodiments of the present technology.

FIG. 4 is a flow diagram of an example process for identifying a treatment location in accordance with the present technology.

FIG. 5 is a flow diagram of an example process for obtaining data characterizing a swallowing function parameter in accordance with the present technology.

### DETAILED DESCRIPTION

Delivery of electrical stimulation energy to afferent and sensory nerves in the pharynx can produce changes in motor cortical representation and cortical excitability that are associated with improvements in swallowing function. The position at which electrical stimulation energy is delivered is essential to the efficacy and safety of pharyngeal electrical stimulation (PES). For example, if the conductive elements are positioned too far superiorly within the pharynx, the trigeminal nerve and/or the facial nerves may be stimulated, which may in turn cause undesirable off-target effects such as facial pain or jaw chattering. If the conductive elements are positioned too far inferiorly, delivery of the electrical stimulation energy may unintentionally modify a patient's cardiac activity and/or motor activity of the patient's upper esophageal sphincter. Moreover, because the density, depth, and/or types of nerves vary at different locations along the upper respiratory and gastrointestinal tracts, delivering energy at one location can be highly efficacious while delivering energy at another location as close as a centimeter away from the first location can provide only marginal therapeutic benefits. For example, different sensory nerves and cortical regions are involved in the esophageal and pharyngeal phases of swallowing. Accordingly, if the conductive elements are positioned in the esophagus, the therapeutic benefit of treating oropharyngeal dysphagia via electrical stimulation may be limited. Additionally or alternatively, if the conductive elements are positioned too far inferiorly, it may be challenging to obtain sufficient contact between the conductive elements and the pharyngeal mucosa, which may limit efficacy of the treatment.

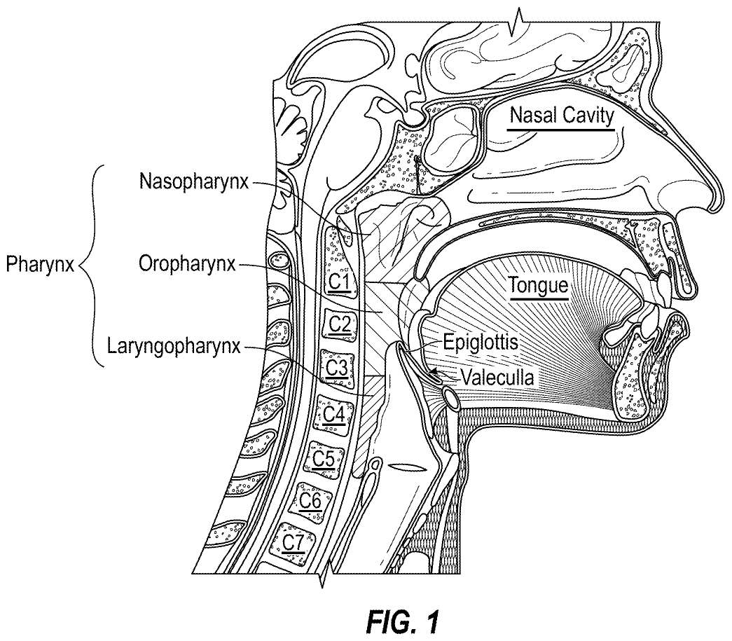

To address the foregoing challenges, several embodiments of the present technology are related to devices, systems, and methods for identifying a treatment location within the pharynx for delivering electrical stimulation energy. In some embodiments, the treatment location is a location that, when stimulated, is associated with an increase in motor activity and/or functional reorganization of the cortex. Such methods can include applying electrical stimulation energy at one or more locations in a pharynx of a human subject, obtaining data characterizing a swallowing function parameter of the subject after applying the stimulation energy at each of the one or more locations, and based on the data, identifying a preferred treatment location. Specific details of several embodiments of the technology are described below with reference to FIGS. 1-5.

### I. Anatomy and Physiology

The pharynx is the part of the digestive system situated posterior to the nasal and oral cavities and posterior to the larynx. It is therefore divisible into nasal, oral, and laryngeal parts: the (1) nasopharynx, (2) oropharynx, and (3) laryngopharynx. With reference to FIG. 1, the pharynx extends from the base of the skull down to the inferior border of the cricoid cartilage (around the C6 vertebral level), where it becomes continuous with the esophagus. Its superior aspect is related to the sphenoid and occipital bones and the posterior aspect to the prevertebral fascia and muscles as well as the upper six cervical vertebrae. The pharynx is a fibromuscular tube lined by mucous membrane.

The pharynx is the common channel for deglutition (swallowing) and respiration, and the food and air pathways cross each other in the pharynx. In the anesthetized patient, the passage of air through the pharynx is facilitated by extension of the neck.

The nasopharynx, at least in its anterior part, may be regarded as the posterior portion of the nasal cavity, with which it has a common function as part of the respiratory system. The nasopharynx communicates with the oropharynx through the pharyngeal isthmus, which is bounded by the soft palate, the palatopharyngeal arches, and the posterior wall of the pharynx. The isthmus is closed by muscular action during swallowing. The choanae are the junction between nasopharynx and the nasal cavity proper.

A mass of lymphoid tissue, the nasopharyngeal tonsil is embedded in the mucous membrane of the posterior wall of the nasopharynx. Enlarged nasopharyngeal tonsils are termed “adenoids” and may cause respiratory obstruction. Higher up, a minute pharyngeal hypophysis (resembling the adenohypophysis) may be found.

The oropharynx extends inferiorward from the soft palate to the superior border of the epiglottis. It communicates anteriorly with the oral cavity by the faucial (oropharyngeal) isthmus, which is bounded superiorly by the soft palate, laterally by the palatoglossal arches, and inferiorly by the tongue. This area is characterized by a lymphatic ring composed of the nasopharyngeal, tubal, palatine, and lingual tonsils.

The mucous membrane of the epiglottis is reflected onto the base of the tongue and onto the lateral wall of the pharynx. The space on each side of the median glosso-epiglottic fold is termed the epiglottic vallecula.

The laryngopharynx extends from the superior border of the epiglottis to the inferior border of the cricoid cartilage, where it becomes continuous with the esophagus. Its anterior aspect has the inlet of the larynx and the posterior aspects of the arytenoid and cricoid cartilages. The pyriform sinus, in which foreign bodies may become lodged, is the part of the cavity of the laryngopharynx situated on each side of the inlet of the larynx.

A. Muscles

The pharynx consists of four coats of muscles, from within outward: (1) a mucous membrane continuous with that of the auditory tubes and the nasal, oral, and laryngeal cavities; (2) a fibrous coat, that is thickest in its superior extent (pharyngobasilar fascia) and that forms a median raphe posteriorly; (3) a muscular coat, described below; and (4) a fascial coat (buccopharyngeal fascia) covering the outer surface of the muscles.

The wall of the pharynx is composed mainly of two layers of skeletal muscles. The external, circular layer comprises three constrictors. The internal, chiefly longitudinal layer consists of two levators: the stylopharyngeus and the palatopharyngeus.

The chief action in which the muscles of the pharynx combine is deglutition (or swallowing), a complicated, neuromuscular act whereby food is transferred from (1) the mouth through (2) the pharynx and (3) the esophagus to the stomach. The pharyngeal stage is the most rapid and most complex phase of deglutition. During swallowing, the nasopharynx and vestibule of the larynx are sealed but the epiglottis adopts a variable position. Food is usually deviated laterally by the epiglottis and ary-epiglottic folds into the piriform recesses of the laryngopharynx, lateral to the larynx. The pharyngeal ridge is an elevation or bar on the posterior wall of the pharynx inferior to the level of the soft palate; it is produced during swallowing by transverse muscle fibers.

B. Innervation and Blood Supply

The motor and most of the sensory supply to the pharynx is by way of the pharyngeal plexus, which is formed by the pharyngeal branches of the vagus and glossopharyngeal nerves and also by sympathetic nerve fibers. The motor fibers in the plexus are carried by the vagus (although they likely represent cranial accessory nerve components) and supply all the muscles of the pharynx and soft palate except the stylopharyngeus (supplied by cranial nerve IX) and tensor veli palatini (supplied by cranial nerve V). The sensory fibers in the plexus are from the glossopharyngeal nerve, and they supply the greater portion of all three parts of the pharynx. The pharynx is supplied by branches of the external carotid (ascending pharyngeal) and subclavian (inferior thyroid) arteries.

### II. Select Embodiments of Pharyngeal Electrical Stimulation Devices

Without being bound by theory, it is believed that increasing peripheral (sensory) feedback to higher brain centers improves the local sensory performance. Many processes in the body rely on a combination of local (peripheral) and centralized neurological control and feedback to allow multiple nerves and muscles to combine and coordinate correctly to execute a functional activity. Examples of such processes include swallowing and speech, as well as processes such as urinary/fecal control and gastric motility. Afferent nerve signals from peripheral nerves provide contextual information about the activity in question. This information is processed centrally and the outputs are efferent signals that then modulate the activity of the muscles. After brain injury or as a consequence of persistent disuse, areas in the brain responsible for coordination or control can become dysfunctional. The absence of ‘neurological oversight’ associated with such dysfunction means that although local nerves and muscles are not damaged, they are no longer capable in isolation of delivering correct functional performance. When applied therapeutically, electrical stimulation at appropriate frequencies, intensities, and locations can trigger afferent nerve signals from peripheral sensory receptors. The stimulation conditions used and the afferent signals arising are designed to target and maximally excite specific areas in the brain associated with known functional processes (e.g., the swallow motor cortex). The excitation is associated with a neuroplastic functional reorganization that can restore central processing capability.

The devices of the present technology are configured to stimulate sensory nerves in the pharynx, thereby inducing and accelerating a cortical reorganization process whereby responsibility for the control and coordination of motor activity is moved from a damaged area of the brain to a complementary area of the cortical centers of the brain with intact function. It is also believed that the stimulation energy of the present technology can increase excitability of the motor cortex and/or increase local levels of specific neurotransmitters in the pharynx and/or the esophagus to facilitate the motor activity.

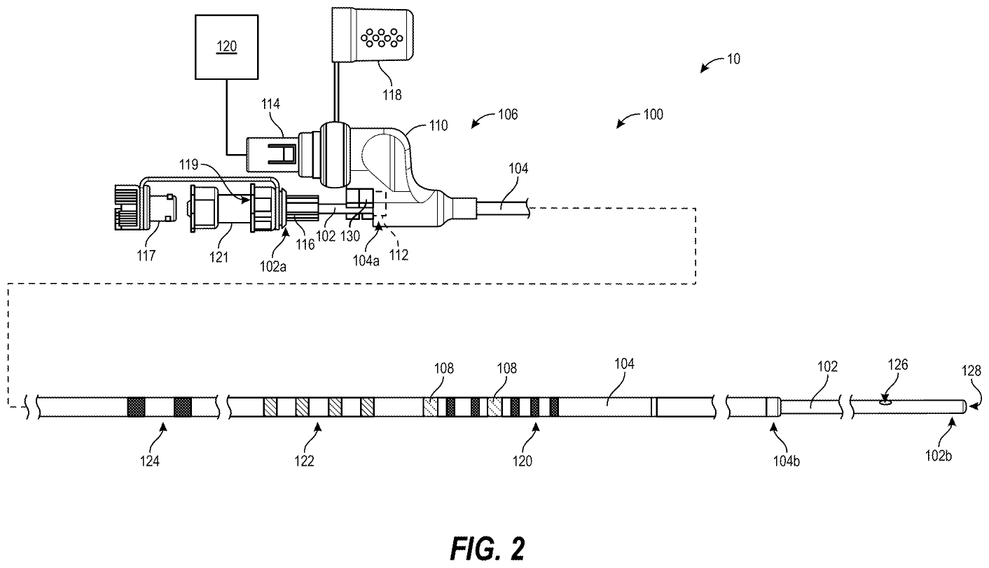

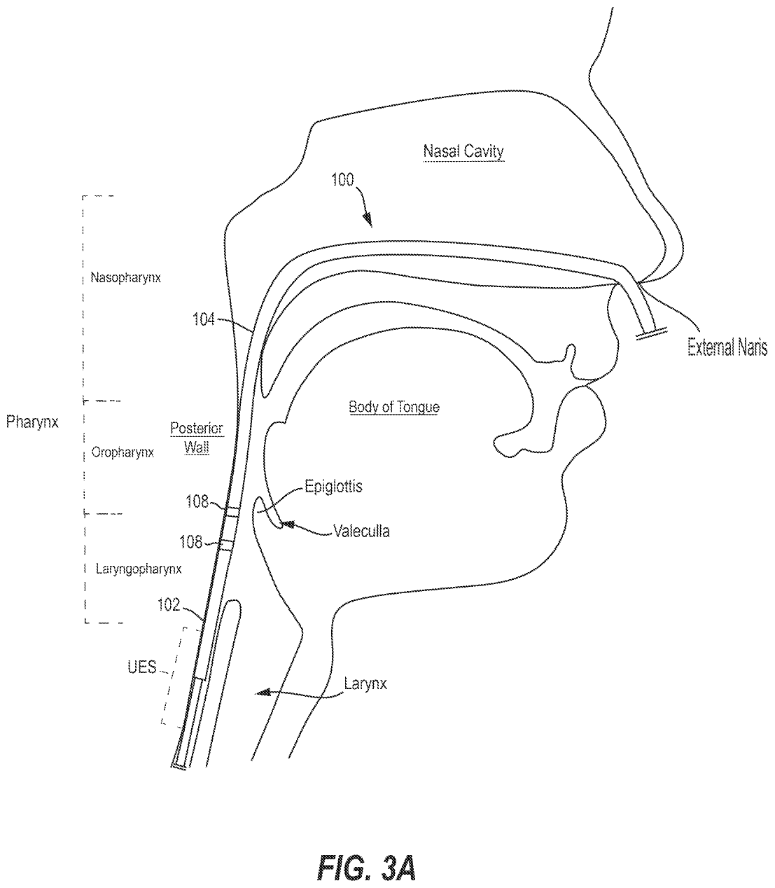

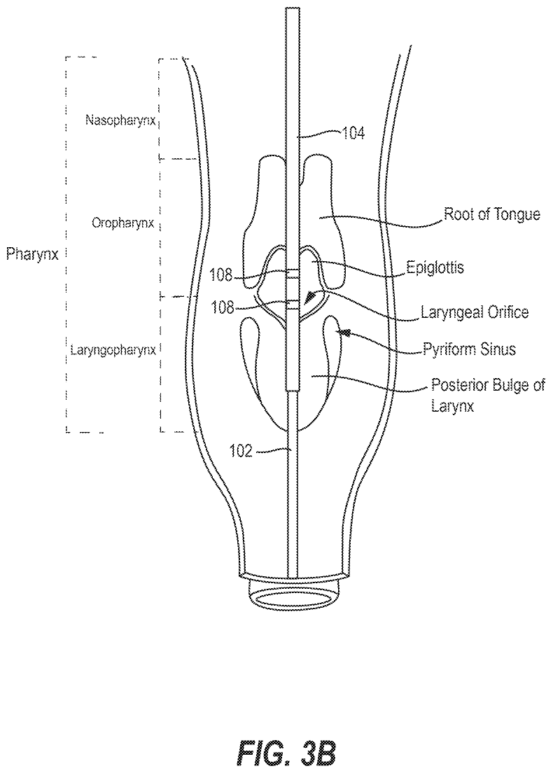

FIG. 2 depicts a treatment system 10 configured in accordance with several embodiments of the present technology. The system 10 may comprise a device 100 configured to provide intraluminal electrical stimulation to a patient suffering from a medical condition, and a current generator 120 configured to be electrically coupled to the device 100. The device 100 can include a handle assembly 106, a first elongated shaft 102 (or “first shaft 102”), and a second elongated shaft 104 (or “second shaft 104”) configured to slidably receive the first shaft 102 therethrough. The first shaft 102 can be a flexible tube configured to deliver nutrients to the patient. For example, in some embodiments the first shaft 102 comprises a nasogastric feeding tube. Each of the first and second shafts 102, 104 have a proximal portion 102a and 104a, respectively, coupled to the handle assembly 106, and a distal portion 102b and 104b, respectively, configured to be positioned within an upper gastrointestinal tract of the patient. As depicted in FIGS. 3A and 3B, when the device 100 is inserted into the patient, the distal portion 102b of the first shaft 102 is configured to be positioned within the patient's stomach, and the distal portion 104b of the second shaft 104 is configured to be positioned at a treatment location within the patient's pharynx. The device 100 further includes one or more conductive elements 108 carried by the distal portion 104b of the second shaft 104 and configured to deliver stimulation energy to nerves proximate the treatment location.

As shown in FIG. 2, the handle assembly 106 can include a hub 110 and one or more connectors and/or accessory components configured to be removably coupled to the hub 110. The hub 110 can include a housing having a first portion fixedly coupled to the proximal portion 104a of the second shaft 104, and a second portion configured to house one or more electrical components. In some embodiments, the second portion of the housing is positioned laterally of the first portion of the housing. The hub 110 may further include an electrical connector 114 at the second portion that provides an electrical interface between the second shaft 104 and the current generator 120.

In some embodiments, the device 100 includes a connector 116 coupled to the proximal portion 102a of the first shaft 102 and having one or more ports, such as port 119, configured to be coupled to one or more accessory devices or systems. For example, the port 119 may be configured to be releasably coupled to an enteral feeding set (not shown) for delivering nutrients through the first elongated shaft 102 into the patient's stomach. Additionally or alternatively, the port 119 can be configured to be releasably coupled to a guidewire assembly. For example, the port 119 can be configured to receive a guidewire (not visible) therethrough to assist with inserting the device 100 into the patient. The guidewire assembly can include a guidewire grip 121 coupled to the proximal end portion of the guidewire. In some embodiments, the connector 116 includes one or more additional ports, such as a port configured to be fluidly coupled to a syringe or other fluid source and/or pressure source. The connector 116 may further comprise a cap 117 tethered to the connector 116 and configured to be secured over the port 119 when not in use.

As previously mentioned, the first shaft 102 is configured to be inserted through a lumen of the second shaft 104. In use, the distal portion 102b of the first shaft 102 can be inserted into an opening at the proximal portion 104a of the second shaft 104 that is fixed to the hub 110. In some embodiments, the device 100 includes a sealing member (not visible) at the hub 110 that engages the first and second shafts 102, 104 to prevent fluid from within a patient being drawn up within a space between the first and second shafts 102, 104 by way of capillary action when the second shaft 104 is removed from the patient. The sealing member may also be configured to clean any matter off of the first shaft 102 as it is withdrawn from the patient.

The first shaft 102 can have an atraumatic distal tip for patient comfort and ease of inserting the first shaft 102 into the patient. The first shaft 102 can have an opening 128 at its distal end and/or one or more apertures 126 extending through the sidewall along the distal portion 102b of the first shaft 102. Nutrients can be dispersed from the first shaft 102 into the patient's stomach via the one or more apertures 126 and/or the opening 128.

Each of the conductive elements 108 may comprise an electrode, an exposed portion of a conductive material, a printed conductive material, and other suitable forms. In some embodiments, for example as shown in FIG. 2, the conductive elements 108 comprise a pair of ring electrodes configured to deliver bipolar stimulation energy. The conductive elements 108 can be crimped, welded, or otherwise adhered to an outer surface of the second shaft 104. In some embodiments, the conductive elements 108 comprise a flexible conductive material disposed on the second shaft 104 via printing, thin film deposition, or other suitable techniques. While the device 100 shown in FIG. 2 includes two conductive elements 108, in some embodiments the device 100 may include more or fewer than two conductive elements 108. For example, the device 100 may comprise a single conductive element 108 configured to generate a monopolar electric field. Such embodiments include a neutral or dispersive electrode electrically connected to the current generator 120 and positioned on the patient's skin. In some embodiments, the device 100 may include three or more conductive elements 108 spaced apart along a longitudinal axis of the second shaft 104.

The device 100 may include one or more conductive leads (not visible) extending between a proximal portion of the device 100, such as the hub 110, and the conductive elements 108. In some embodiments, for example, the conductive leads comprise two wires, each extending distally from the hub 110 through a channel (the same channel or different channels) in the second shaft 104 to a corresponding one of the conductive elements 108. The channel(s), for example, can extend longitudinally within a sidewall of the shaft 104. The conductive leads can be insulated along all or a portion of their respective lengths.

In some embodiments, the device 100 is configured such that a position of the second shaft 104 can be fixed relative to a position of the first shaft 102 (or vice versa). The position of the first shaft 102 relative to the second shaft 104 may be adjusted prior to insertion of the device 100 into the patient. Once adjustment is complete, the relative positions of the first shaft 102 and second shaft 104 may be substantially fixed. For example, as shown in FIG. 2, the proximal portion 100a of the device 100 can include a retaining structure 130 configured to be coupled to the hub 110 and/or the proximal portions 102a, 104a of one or both of the second shaft 104 and the first shaft 102. The retaining structure 130 can have a first portion on or through which the second shaft 104 and/or first shaft 102 may be movably or fixedly positioned, and a second portion moveable relative to the first portion. When the second portion is in an open position (as shown in FIG. 1), the first shaft 102 and the second shaft 104 can move longitudinally relative to one another. When the second portion is in a closed position (not shown), the longitudinal and/or radial positions of the first shaft 102 and the second shaft 104 are substantially fixed relative to one another.

The retaining structure 130 may be fixed to one of the first shaft 102 or the second shaft 104 and, at least in the open configuration, allow movement of the other of the first shaft 102 and the second shaft 104. In some embodiments, for example as shown in FIG. 2, the proximal portion of the second shaft 104 is fixed to the retaining structure 130 and the proximal portion of the first shaft 102 is slidably received by the first portion when the device 100 is assembled and the second portion is in an open position. When the second portion is in a closed position, the second portion engages the first shaft 102 and fixes the first shaft 102 relative to the second shaft 104, the hub 110, and/or the retaining structure 130.

In any case, the portion of the retaining structure 130 configured to engage the first and/or second shafts 102, 104 can comprise a high friction thermoplastic elastomer liner that engages the proximal portion of the first shaft 102 when the second portion is in the closed position. The liner can be configured to reduce the compressive force required to fix the first shaft 102 and thereby prevent pinching of the first shaft 102. Other suitable shapes, materials, positions, and configurations for the retaining structure 130 are possible. For example, the retaining structure 130 can comprise one or more magnets, a screw and threaded insert, a radial compression clip, and/or others to fix the proximal portion of the first shaft 102 to the retaining structure 130, the hub 110, and/or the second shaft 104.

As previously mentioned, the proximal portion of the device 100 and/or second shaft 104 is configured to be electrically coupled to a current generator 120 for delivering electric current to the conductive elements 108. The current generator 120, for example, can include a power source and a controller. The controller includes a processor coupled to a memory that stores instructions (e.g., in the form of software, code or program instructions executable by the processor or controller) for causing the power source to deliver electric current according to certain parameters provided by the software, code, etc. The power source of the current generator 120 may include a direct current power supply, an alternating current power supply, and/or a power supply switchable between a direct current and an alternating current. The current generator 120 can include a suitable controller that can be used to control various parameters of the energy output by the power source or generator, such as intensity, amplitude, duration, frequency, duty cycle, and polarity. Instead of or in addition to a controller, the current generator can include drive circuitry. In such embodiments, the current generator can include hardwired circuit elements to provide the desired waveform delivery rather than a software-based generator. The drive circuitry can include, for example, analog circuit elements (e.g., resistors, diodes, switches, etc.) that are configured to cause the power source to deliver electric current according to the desired parameters. For example, the drive circuitry can be configured to cause the power source to deliver periodic waveforms. In some embodiments, the drive circuitry can be configured to cause the power source to deliver a unipolar square wave.

The current generator 120 may be configured to provide a stimulation energy to the conductive elements 108. To treat a specific condition, the current generator 120 may be configured to provide a stimulation energy that has an intensity, amplitude, duration, frequency, duty cycle, and/or polarity such that the conductive elements 108 apply an electric field at the treatment location that promotes neuroplasticity in specific areas of the brain. The controller can automatically vary the voltage (to a maximum of 250V) in order to deliver the set current. In some embodiments, the only user adjustable parameter is the stimulation intensity which is derived during treatment level optimization prior to every treatment. Patient specific threshold levels are determined by establishing a sensory threshold followed by measurement of the maximum tolerated stimulation intensity. The controller may automatically calculate the correct stimulation level from the sensory threshold and maximum tolerated stimulation intensity and sets this as the output. The current generator 120 can provide, for example, a current of about 1 mA to about 50 mA, about 1 mA to about 40 mA, about 1 mA to about 30 mA, about 1 mA to about 20 mA, or about 1 mA to about 10 mA, at a frequency of about 1 Hz to about 50 Hz, about 1 Hz to about 40 Hz, about 1 Hz to about 30 Hz, about 1 Hz to about 20 Hz, about 1 Hz to about 10 Hz, about 2 Hz to about 8 Hz, about 1 Hz, about 2 Hz, about 3 Hz, about 4 Hz, about 5 Hz, about 6 Hz, about 7 Hz, about 8 Hz, about 9 Hz, or about 10 Hz, and having a pulse width of about 150 μS to about 250 μS, about 175 μS to about 225 μS, or about 200 μS.

The current generator 120 may also be configured to monitor contact quality between the conductive elements 108 and patient tissue during treatment set up/optimization and throughout the treatment process. In some embodiments, the current generator 120 records and stores patient information and includes a USB port to enable downloading of patient data. The current generator may include a touch screen user interface and software to guide a user through the treatment process.

When the conductive elements 108 are in a desired treatment position to treat a condition, stimulation energy can be delivered to the treatment location via the conductive elements 108. In some embodiments, the delivered current is a unipolar square wave having an amplitude between 1 mA and 50 mA, a frequency of 5 Hz, and a pulse duration of 200 μS. Each treatment session can have a duration between 5 minutes and 20 minutes. For example, the treatment session can have a duration of 10 minutes. In some embodiments, a patient can undergo a single treatment per day over the course of multiple days of treatment. For example, a patient can undergo one treatment session per day for three to six consecutive days. In some embodiments, the patient may undergo multiple treatment sessions per day and/or per week. Still, other current parameters and treatment parameters are possible.

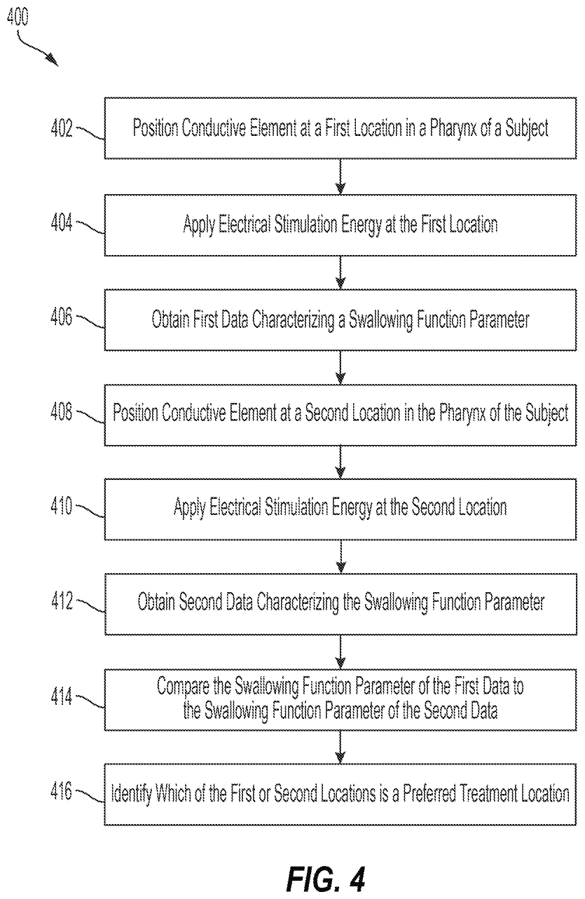

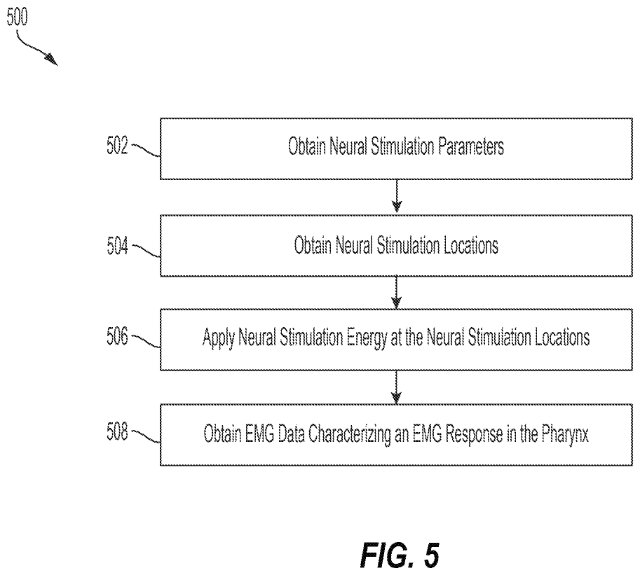

As shown in FIGS. 3A and 3B, in some embodiments each of the conductive elements 108 can be configured to be positioned at a treatment location 300 within a treatment window 302. In some embodiments, the treatment window can be positioned along the oropharynx and/or the laryngopharynx. For example, the conductive elements 108 can be positioned at treatment locations 300 within a treatment window 302 that is at or adjacent to the junction between the C3 and C4 cervical vertebrae. However, the treatment locations can be at any suitable position within the pharynx. As described in greater detail herein with reference to FIGS. 4 and 5, the treatment location(s) and/or the treatment window can be identified according to any of the methods disclosed herein. For example, the treatment location(s) and/or treatment window may be selected based on an evaluation and comparison of multiple potential treatment locations. In some embodiments, the treatment location(s) correspond to positions demonstrating a favorable increase in cortical excitability and/or functional reorganization of the cortex when electrical stimulation energy is applied thereto.

In some embodiments, the first and/or second shafts 102, 104 can comprise one or more indicators configured to facilitate insertion and positioning of the device 100 within the patient. For example, the indicator can comprise one or more visual markings that, when viewed through the patient's oral cavity, indicate the conductive elements 108 are properly positioned at the desired treatment location or that the second shaft 104 (and/or conductive elements 108) should be inserted further or withdrawn. In some embodiments, the indicator comprises one or more circumferential markings (such as one or more colored bands) printed on the second shaft 104.

### III. Electrical Stimulation Treatment Locations

The conductive elements of a stimulation device can each be configured to be positioned at a treatment location within a lumen of the pharynx such that, when energy is delivered to the conductive elements, the conductive elements emit an electric field covering the pharynx, the base of the tongue, the epiglottis, the region above the larynx, and/or another oral, nasal, or esophageal tissue. Each of these regions includes a high density of sensory nerves, including the pharyngeal plexus, the superior laryngeal branch of the vagus nerve, the lingual branch of the glossopharyngeal nerve, the internal branch of the superior laryngeal nerve, and/or the external branch of the superior laryngeal nerve. Delivery of the electrical stimulation energy to the nerves activates the nerves such that afferent information is transmitted to the nucleus tractus solitarius (NTS) within the brainstem. The NTS processes incoming afferent information, communicates with the cortex and subcortex, and communicates with an efferent network of neurons around the nucleus ambiguus. Thus, at a brainstem level there is communication between sensory inputs and coordinated motor outputs. Moreover, intercortical connections may enable direct communication between sensory and motor cortical centers. It is through these lower level and higher level interconnections that electrical stimulation of the pharynx is thought to influence cortical motor outflow.

In some embodiments, electrical stimulation of the pharynx can be employed to treat a medical condition. For example, PES can be employed to treat dysphagia. As previously noted, dysphagia is a condition in which a patient has difficulty in swallowing or is unable to swallow safely. Swallowing is a complex sensorimotor activity with voluntary and involuntary components, reflecting central regulatory pathways within swallowing centers in the cortex and brainstem, respectively. Electrical stimulation energy can be delivered to nerves proximate the pharynx at a treatment location and/or treatment window to modulate cortical representation and/or cortical excitability associated with the muscles of the pharynx, and thereby modulate swallowing function. A cortical representation associated with the muscles of the pharynx can comprise an area of the motor cortex that, when stimulated, elicits an electromyographic (EMG) response in the pharynx. Additionally or alternatively, a cortical excitability associated with the muscles of the pharynx can comprise an EMG response to stimulation of the cortex that is measured in the pharynx. In some embodiments, the EMG response characterizes a motor-evoked potential (MEP) parameter such as an amplitude or latency of the EMG response.

As previously noted, the position at which electrical stimulation energy is delivered within the pharynx is essential to the efficacy and safety of PES. For example, if the conductive elements are positioned too far superiorly within the pharynx, the trigeminal nerve or the facial nerves may be stimulated, which may in turn cause undesirable off-target effects such as facial pain or jaw chattering. If the conductive elements are positioned too far inferiorly, delivery of the electrical stimulation energy may unintentionally modify a patient's cardiac activity and/or motor activity of the patient's upper esophageal sphincter. Moreover, because the density, depth, and/or types of nerves vary at different locations along the upper respiratory and gastrointestinal tracts, delivering energy at one location can be highly efficacious while delivering energy at another location as close as a centimeter away from the first location can provide only marginal therapeutic benefits. For example, different sensory nerves and cortical regions are involved in the esophageal and pharyngeal phases of swallowing. Accordingly, if the conductive elements are positioned in the esophagus, the therapeutic benefit of treating oropharyngeal dysphagia via electrical stimulation may be limited. Additionally or alternatively, if the conductive elements are positioned too far inferiorly, it may be challenging to obtain sufficient contact between the conductive elements and the pharyngeal mucosa, which may limit efficacy of the treatment.

Thus, some embodiments of the present technology are directed to a method of identifying a treatment location for delivering stimulation energy to treat a medical condition. In some embodiments, the treatment location comprises a location within a pharynx of a patient that a conductive element is configured to be positioned at or adjacent to. In various embodiments, the method can comprise identifying a treatment window spanning a specific distance that one or more conductive elements are configured to be positioned within. The method can comprise applying electrical stimulation energy at one or more locations in the pharynx of a subject and, during and/or after applying the energy at one or more of the locations, evaluating one or more parameters associated with the subject's swallowing function. In some embodiments, the preferred treatment location is the location which, when stimulated, produces the greatest change in the swallowing function parameter(s) and/or the most favorable swallowing function parameter(s).

FIG. 4 is a flow diagram of an example process 400 for identifying a treatment location for PES in accordance with several aspects of the present technology. As described in greater detail herein, the process 400 can include positioning a conductive element at one or more potential treatment locations in a pharynx of a subject, applying electrical stimulation energy at each of the potential treatment locations, and obtaining data characterizing a swallowing function parameter of the subject associated with stimulation of each potential treatment location. The data characterizing the swallowing function parameter can be obtained prior to, during, and/or after applying the electrical stimulation energy at a potential treatment location. As described in greater detail herein with reference to FIGS. 4 and 5, obtaining the data can include stimulating a neural tissue of the subject and obtaining a pharyngeal EMG response to the stimulation. In some embodiments, the process 400 includes comparing the swallowing function parameters associated with each potential treatment location and, based on the comparison, identifying which of the potential treatment locations is a preferred treatment location for delivering electrical stimulation energy to treat a medical condition.

Any of the processes disclosed herein can be performed with a group of human subjects. Accordingly, the process 400 can identify a preferred treatment location that is associated with the most favorable swallowing function parameter and/or change in swallowing function parameter amongst the group of subjects. Additionally or alternatively, any of the processes disclosed herein can be performed with a single subject such that a subject specific preferred treatment location is identified. The human subject(s) may or may not be currently or previously suffering from dysphagia.

As shown in FIG. 4, the process 400 can begin at block 402 with positioning a conductive element in a pharynx of a human subject at a first location. In some embodiments, the conductive element is carried by an elongate member. The conductive element can be similar to the conductive elements 108 shown in FIGS. 2-3B and/or the elongate member can be similar to the first shaft 102 and/or second shaft 104 shown in FIGS. 2-3B. Additionally or alternatively, the conductive element can be wholly or partially different from the conductive elements 108 and/or the elongate member can be wholly or partially different from the first and/or second shafts 104. The conductive element can comprise one conductive element, two conductive elements, or three or more conductive elements. In some embodiments, the conductive element comprises a ring electrode, printed conductive material, or any other suitable conductive element.

According to various embodiments, the conductive element and the elongate member are positioned within a lumen of the subject's pharynx such that the conductive element is positioned at the first location. The elongate member and/or conductive element can be inserted into the lumen of the pharynx of the subject via a nasal cavity of the subject (see FIGS. 3A and 3B, for example) or an oral cavity of the subject. Additionally or alternatively, the elongate member and/or conductive element can be inserted into the lumen the pharynx of the subject via transcutaneous, transmuscular, and/or percutaneous insertion. In various embodiments, one or more of the conductive elements is extracorporeally positioned.

The first location can be positioned along the nasopharynx, the oropharynx, and/or the laryngopharynx. In some embodiments, the first location can be positioned within the esophagus, the trachea, the oral cavity, or the nasal cavity of the subject. The first location can be proximate an upper esophageal sphincter (UES) of the subject. However, as the UES is a muscular structure, it may be advantageous to space the conductive element apart from the UES to prevent or limit noise due to motor activity of the UES in EMG responses obtained at the first location. For example, the conductive element may be spaced apart from the UES by less than 1 cm, about 1 cm, about 2 cm, about 3 cm, about 4 cm, or 5 cm or more.

In some embodiments, positioning the conductive element at the first location can comprise evaluating a position of the conductive element once the elongate member has been inserted and, optionally, repositioning the conductive element until the conductive element is positioned at the first location. In some embodiments, the elongate member and/or the conductive element comprises one or more indicators configured to facilitate positioning of the conductive element at an intended location (e.g., the first location, etc.). For example, the indicator can comprise one or more visual markings that, when viewed through the patient's oral cavity, indicate if the conductive element is properly positioned or if the elongate member and/or conductive element should be inserted further or withdrawn. In some embodiments, the indicator comprises one or more circumferential markings (such as one or more colored bands) on the elongate member.

Additionally or alternatively, the elongate member can include one or more sensors configured to facilitate positioning the conductive element at an intended location (e.g., the first location, etc.). For example, the elongate member can include a strain gauge transducer configured to indicate when the conductive element is at the intended location by detecting the pressure in the pharynx. The strain gauge transducer can be positioned on the elongate member distal of the conductive element such that, when the strain gauge transducer is positioned in the UES, the pressure detected by the strain gauge transducer increases and thereby indicates that the conductive element is at the intended location. In some embodiments, the strain gauge transducer can be positioned between two conductive elements, adjacent to one conductive element, etc. Additionally or alternatively, the one or more sensors can comprise any suitable sensing modality (e.g., pressure, inductive, pH, etc.).

The process 400 can proceed at block 404 with applying electrical stimulation energy at the first location via the conductive element. As previously described with reference to FIG. 2, in some embodiments a current generator is configured to provide the electrical stimulation energy to the conductive element. The current generator may be configured to provide electrical stimulation energy that has an intensity, amplitude, duration, frequency, duty cycle, and/or polarity such that the conductive element applies an electric field configured to promote neuroplasticity. In some embodiments, an intensity of the electrical stimulation energy can be the same for each human subject or the intensity can be subject specific. For example, a subject specific sensory threshold and/or a subject specific maximum tolerated stimulation intensity can be determined and/or applied.

The current generator can provide, for example, a current of about 1 mA to about 50 mA, about 1 mA to about 40 mA, about 1 mA to about 30 mA, about 1 mA to about 20 mA, or about 1 mA to about 10 mA, at a frequency of about 1 Hz to about 50 Hz, about 1 Hz to about 40 Hz, about 1 Hz to about 30 Hz, about 1 Hz to about 20 Hz, about 1 Hz to about 10 Hz, about 2 Hz to about 8 Hz, about 1 Hz, about 2 Hz, about 3 Hz, about 4 Hz, about 5 Hz, about 6 Hz, about 7 Hz, about 8 Hz, about 9 Hz, or about 10 Hz, and having a pulse width of about 1500 to about 250μS, about 1750 to about 225μS, or about 200 μS. In some embodiments, the delivered current is a unipolar square wave having an amplitude between 1 mA and 50 mA, a frequency of 5 Hz, and a pulse duration of 200 μS. Application of the electrical stimulation energy can have a duration between 5 minutes and 20 minutes.

As shown at block 406 in FIG. 4, the process 400 can include obtaining first data characterizing a swallowing function parameter of the subject. The process 400 can include obtaining the first data during and/or after applying the electrical stimulation energy at the first location. Although not depicted in FIG. 4, the process 400 can include obtaining first baseline data characterizing the swallowing function parameter prior to applying the electrical stimulation energy at the first location. According to various embodiments, the processes for obtaining the first data and the first baseline data can be the same, either entirely or in part. In some embodiments, the processes for obtaining the first data and the first baseline data can be different.

The swallowing function parameter can comprise a direct or an indirect parameter of a subject's ability to safely swallow. In some embodiments, the swallowing function parameter comprises a cortical representation and/or a cortical excitability associated with the pharynx. Additionally or alternatively, the swallowing function parameter can comprise an inter-swallow interval, a swallowing speed, a swallowing capacity, a medical image, a measure of blood flow, an electrical signal, a chemical signal, a mechanical signal, an outcome measure score, an outcome measure interpretation or assessment, an opinion of a clinician, and/or other suitable parameters of swallowing function. Obtaining data (e.g., first baseline data, first data, etc.) characterizing the swallowing function parameter can include performing an instrumental evaluation such as, but not limited to, functional magnetic resonance imaging (fMRI), magnetic resonance imaging (MM), positron emission tomography (PET), magnetoencephalography (MEG), videofluoroscopy (VFS), fiberoptic endoscopic evaluation of swallowing (FEES), pharyngeal manometry, near-infrared spectroscopy (NIRS), functional near-infrared spectroscopy (fNIRS), electroencephalography (EEG), electromyography (EMG), combinations thereof, and/or others. In some embodiments, obtaining the data comprises administering an outcome measure such as, but not limited to, a self-report outcome measure, a performance-based measure, an observer-reported measure, and/or a clinician-reported measure. For example, obtaining the data can comprise administering the functional oral intake scale (FOIS), the dysphagia severity rating scale (DSRS), the dysphagia outcome and severity scale (DOSS), and/or another suitable outcome measure. Additionally or alternatively, obtaining the data can comprise performing a bedside clinical assessment such as a water swallow test, a repetitive saliva swallowing test (RSST), a colored water test, a swallowing provocation test, and/or another suitable bedside clinical assessment. In some embodiments, obtaining the data comprises receiving the data after one or more suitable processes (e.g., performing an instrumental evaluation, administering an outcome measure, performing a bedside clinical assessment, etc.) have been performed. For example, obtaining the data can comprise receiving fMRI data from an entity (e.g., a hospital, a medical imaging facility, etc.) responsible for performing the fMRI evaluation. Additionally or alternatively, obtaining the data can comprise generating the data.

As previously noted, the swallowing function parameter can comprise a cortical representation and/or a cortical excitability associated with the pharynx. Cortical representation and cortical excitability can be associated with swallowing function, as evidenced, for example, by enlargement in cortical representation associated with the pharynx in an undamaged hemisphere of a patient's brain when the other hemisphere has been damaged by stroke or other neurological injury. A cortical representation associated with the pharynx can correspond to an area of the cortex that, when stimulated, elicits an EMG response in the pharynx. In some embodiments, a cortical excitability corresponds to an MEP parameter (e.g., an amplitude, a latency, etc.) of an EMG response to stimulation of the cortex that is detected in the pharynx. As described in greater detail with reference to FIG. 5, obtaining data (e.g., baseline data, first data, etc.) characterizing a cortical representation and/or a cortical excitability can comprise applying neural stimulation energy a subject's neural tissue (e.g., by transcranial magnetic stimulation (TMS)) and obtaining EMG data characterizing an EMG response within the pharynx.

The process 400 can include applying electrical stimulation energy at multiple locations in the pharynx and evaluating a swallowing function parameter associated with application of the electrical stimulation energy at each of the locations For example as shown in FIG. 4, the process 400 can include positioning the conductive element at a second location in the pharynx of the subject (block 408), applying electrical stimulation energy at the second location (block 410), and obtaining second data characterizing the swallowing function parameter of the subject (block 412). The second location can be spaced apart from the first location. Any of the blocks 408, 410, 412 can be similar to a corresponding block 402, 404, 406 as described with reference to the first location. For example, positioning the conductive element at the second location can comprise evaluating a position of the conductive element and, optionally, repositioning the conductive element until the conductive element is positioned at the second location. Additionally or alternatively, the electrical stimulation energy applied at the second location at block 410 can have the same parameters as the electrical stimulation energy applied at the first location at block 404. Obtaining the second data can be similar to obtaining the first data. In some embodiments, the process 400 includes obtaining second baseline data prior to applying the electrical stimulation energy at the second location.

In some embodiments, obtaining the data (e.g., first data, first baseline data, second data, second baseline data, etc.) characterizing the swallowing function parameter can occur at a specific time with respect to one or more portions of the process 400. For example, obtaining the first data can occur once a specific amount of time has passed after applying the stimulation energy at the first location. Obtaining the first data and/or obtaining the second data can occur immediately after applying the stimulation energy, about 5 minutes or more after applying the stimulation energy, about 15 minutes or more after applying the stimulation energy, about 30 minutes or more after applying the stimulation energy, about 60 minutes or more after applying the stimulation energy, about 90 minutes or more after applying the stimulation energy, about 120 minutes or more after applying the stimulation energy, about 150 minutes or more after applying the stimulation energy, about 180 minutes or more after applying the stimulation energy, about 8 hours or more after applying the stimulation energy, about 12 hours or more after applying the stimulation energy, or about 24 hours or more after applying the stimulation energy.

The process 400 can include comparing the swallowing function parameters of at least two of the first baseline data, the first data, the second baseline data, or the second data. For example, as shown at block 414, the process 400 can include comparing the swallowing function parameter of the first data to the swallowing function parameter of the second data. Additionally or alternatively, the process 400 can include comparing the swallowing function parameter of the baseline first data to the swallowing function parameter of the first data and/or comparing the swallowing function parameter of the baseline second data to the swallowing function parameter of the second data. Comparing the swallowing function parameters can comprise determining a difference between the swallowing function parameters. For example, comparing the swallowing function parameters can include determining a difference in excitability of the regions of the cortex associated with the pharynx between the baseline first data and the first data. In some embodiments, determining a difference in excitability can comprise determining a difference in an MEP parameter, such as an amplitude or latency, obtained from a pharyngeal EMG response. Additionally or alternatively, a topographical map of the cortical representation associated with the pharynx can be obtained to spatially depict the area of the motor cortex that, when stimulated, elicits an EMG response in the pharynx. Comparing the swallowing function parameters can comprise comparing topographical maps obtained from at least two of the first baseline data, the first data, the second baseline data, or the second data. In any of the embodiments disclosed herein, comparing the swallowing function parameters can comprise performing a statistical analysis.

As shown in FIG. 4, the process 400 can include identifying which of the first or second locations is a preferred treatment location for delivering electrical stimulation energy (block 416). In some embodiments, the preferred treatment location is the location associated with a preferred swallowing function parameter. The preferred swallowing function parameter can be the optimal, most favorable, largest, smallest, etc. swallowing function parameter. Additionally or alternatively, the preferred treatment location may be the location associated with a preferred (e.g., optimal, most favorable, largest, smallest, etc.) change in the swallowing function parameter. The change can be evaluated between at least two of the baseline first data, the first data, the baseline second data, or the second data. For example, if an area of a topographical map obtained from the first data is 10% greater than an area of a topographical map obtained from the first baseline data, but an area of a topographical map obtained from the second data is 20% greater than an area of a topographical map obtained from the second baseline data, the second location can be identified as the preferred treatment location.

In some embodiments, identifying the preferred treatment location can comprise performing a weighted analysis of multiple swallowing function parameters. In these and other embodiments, swallowing function parameters with the greatest clinical impact can influence identification of the preferred treatment location to a greater degree than swallowing function parameters with less clinical impact. For example, if a change in cortical representation is weighted higher than a change in MEP latency, the location associated with the greatest change in cortical representation may be identified as the preferred treatment location, even if such location is not associated with the greatest change in MEP latency. In some embodiments, determining the weighting of the swallowing function parameters may involve correlating neurodiagnostic parameters to functional outcome measures and applying greater weighting to neurodiagnostic parameters that are most strongly correlated to functional outcome measures.

Identifying the preferred treatment location can comprise evaluating additional considerations such as off-target effects, practical limitations, and/or other considerations. For example, identifying the preferred treatment location can comprise determining whether application of electrical stimulation energy at the first and/or second locations results in off-target effects such as unintentional motor activity (e.g., jaw chattering, hand twitching, etc.), discomfort, and/or pain. If application of electrical stimulation energy at one of the first and/or second locations results in off-target effects, the location may not be identified as the preferred treatment location. To determine whether application of electrical stimulation energy at the first and/or second locations results in off-target effects, a biological signal (e.g., EMG, ECG, EEG, etc.) can be measured from the subject's face, jaw, hand, heart, and/or other suitable location.

Additionally or alternatively, feedback from the subject and/or an operator performing one or more portions of the process can be obtained to evaluate additional considerations (e.g., off-target effects, practical limitations, etc.) associated with applying electrical stimulation energy at the first and second locations. For example, if an operator struggles to insert the elongate member such that the conductive element is positioned at the first location with sufficient contact between the conductive element and the pharynx, the process may identify that the first location is not the preferred treatment location. Moreover, if a subject experiences pain or discomfort when the conductive element is positioned at one of the first or second locations, the process can identify that the location is not the preferred treatment location.

Although FIG. 4 depicts a process evaluating two potential treatment locations (e.g., the first and second locations), any number of potential treatment locations (e.g., one, two, three, four, five, six, seven, eight, nine, ten, more than ten, etc.) can be evaluated. For example, a predetermined number of potential treatment locations can be evaluated. In some embodiments, the predetermined potential treatment locations can span a certain portion of the pharynx and/or be positioned according to predetermined spacing. In various embodiments, the process 400 can repeat positioning the conductive element at the potential treatment location, applying electrical stimulation energy at the potential treatment location, and obtaining data characterizing a swallowing function parameter of the subject (e.g., blocks 402-406, blocks 408-412) for each of the potential treatment locations. The process 400 can then proceed with comparing the swallowing function parameters associated with each potential treatment location. Additionally or alternatively, the process 400 can comprise comparing the swallowing function parameters associated with two or more potential treatment locations and based on the comparison, identify a preferred treatment location of the two or more potential treatment locations. The process 400 may continue iterating and comparing the swallowing function parameters associated with additional potential treatment locations or may terminate. In some embodiments, the process 400 can terminate once a desired swallowing function parameter has been obtained. For example, the process 400 can terminate once a location associated with a change in cortical excitability greater than a predetermined threshold has been identified.

As previously noted, obtaining data characterizing a swallowing function parameter of a subject (e.g., blocks 406,412, etc.) can comprise obtaining data characterizing a cortical representation and/or a cortical excitability associated with the pharynx. FIG. 5 is a flow chart of an example process 500 for obtaining such data. As will be described in greater detail below, the process 500 can include applying neural stimulation energy to one or more regions of a subject's neural tissue and obtaining EMG data characterizing a pharyngeal EMG response to the stimulation. The EMG data can characterize one or more MEP parameters, such as an amplitude and/or a latency, that are representative of a cortical excitability of the subject. Additionally or alternatively, a cortical representation of the pharynx can be obtained from the EMG data. In some embodiments, the cortical representation comprises a map of those of the one or more regions of the neural tissue that, when stimulated, elicit an EMG response of a certain magnitude in the pharynx.

In some embodiments, applying the neural stimulation energy comprises applying magnetic neural stimulation energy to the one or more regions of the neural tissue via TMS. TMS can produce a magnetic field that penetrates the neural tissue. As the magnetic field passes through the neural tissue, it rapidly decays and induces currents and propagating action potentials within the neural tissue. The resulting action potentials can be detected as motor-evoked potential (MEP) parameters of an EMG response obtained from the muscle controlled by the stimulated neural tissue. For example, applying neural stimulation energy via TMS at the swallowing centers of the cortex of a subject can generate motor activity in the muscles of the pharynx, which can be detected as an EMG response via a conductive element positioned in the pharynx.

According to some embodiments, the process 500 can include obtaining parameters of the neural stimulation energy (block 502). In some embodiments, obtaining the parameters of the neural stimulation energy includes obtaining a threshold intensity corresponding to an intensity at which application of the neural stimulation energy evokes an EMG response above a predetermined threshold. For example, the predetermined threshold of the EMG response in the pharynx can be about 10 μV, at least 10 μV, at least 20 μV, at least 30 μV, at least 40 μV, at least 50 μV, etc. In some embodiments, the neural stimulation energy can have a threshold intensity of about 0.4 T to about 2.4 T, about 0.6 T to about 2.2 T, about 0.8 T to about 2.0 T, about 1.0 T to about 1.8 T, or about 1.2 T to about 1.6 T. In some embodiments, each human subject can have a unique threshold intensity of the neural stimulation energy. Additionally or alternatively, a single threshold intensity of the neural stimulation energy can be identified for use with each subject in a group of human subjects. For example, an average of the unique threshold intensities for a group of subjects can be used for applying neural stimulation energy to all subjects in the group.

As shown in FIG. 5, the process 500 can include obtaining neural stimulation locations at block 504. The neural stimulation locations can be locations that, when stimulated, evoke an EMG response in the pharynx above a predetermined threshold. Obtaining the neural stimulation locations can comprise identifying one or more potential neural stimulation locations, applying neural stimulation energy at the one or more potential neural stimulation locations, and detecting EMG data characterizing a pharyngeal EMG response to the neural stimulation energy for each potential neural stimulation site. The previously-noted process portions can be repeated until all potential neural stimulation locations have been evaluated and/or one or more preferred neural stimulation locations have been identified. The preferred neural stimulation locations can be associated with pharyngeal EMG responses with larger amplitudes and/or lower latencies. In some embodiments, between about 20 and about 150 potential neural stimulation locations can be evaluated. For example, the potential neural stimulation locations can comprise a grid of about 70 potential neural stimulation locations that are spaced apart by approximately 2 cm anteroposteriorly and/or approximately 1 cm mediolaterally. In some embodiments, the most posterior and medial point on grid can be positioned about 2 cm posterior and about 2 cm lateral to the cranial vertex. The grid can be constructed on a flexible sheet and attached closely to the scalp. In some embodiments, when obtaining the neural stimulation locations, the neural stimulation energy can be applied at an intensity of up to about 2.2 T. For example, the neural stimulation energy can be applied at an intensity of about 2.2 T, about 0.4 T to about 2.2 T, about 1.3 T to about 1.8 T, etc.

The neural stimulation locations can comprise one or more regions of the cerebral cortex, including the motor cortex. For example, the neural stimulation sites can comprise one or more of the subject's caudal sensory motor cortex, anterior insula, premotor cortex, frontal operculum, anterior cingulate cortex, prefrontal cortex, anterolateral parietal cortex, posterior parietal cortex, precuneus, superiomedial temporal cortex, posterior cingulate cortex, putamen, and/or caudal nuclei. In some embodiments the neural stimulation locations comprise one or more regions of the swallow centers of the motor cortex.

At block 506, the process 500 can include applying neural stimulation energy at the neural stimulation locations. As previously noted, the neural stimulation energy can comprise magnetic stimulation energy applied via TMS. Applying the neural stimulation energy via TMS can comprise positioning a coil at or adjacent to each of the neural stimulation locations and discharging the coil. In some embodiments, the coil comprises a figure-of-eight coil. The coil can have an outer diameter of between about 20 mm about 100 mm. For example, in some embodiments the coil has an outer diameter of about 70 mm. In some embodiments, the neural stimulation energy can be applied at an intensity greater than a threshold neural stimulation intensity. For example, the neural stimulation energy can be applied at an intensity that is about 0.1 T greater than the threshold intensity, about 0.2 T greater than the threshold intensity, about 0.3 T greater than the threshold intensity, about 0.4 T greater than the threshold intensity, about 0.6 T greater than the threshold intensity, or about 0.7 T or more greater than the threshold intensity. In some embodiments, the neural stimulation energy can be applied at an intensity that is about 105% greater than the threshold intensity, about 110% greater than the threshold intensity, about 115% greater than the threshold intensity, about 120% greater than the threshold intensity, about 125% greater than the threshold intensity, or about 130% greater or more than the threshold intensity. In some embodiments, the neural stimulation energy can be applied to each of the neural stimulation locations once, twice, three times, four times, or more. The process 500 can include waiting a predetermined amount of time between applying the neural stimulation energy at a first neural stimulation location and applying the neural stimulation energy at the first neural stimulation location a subsequent time and/or applying the neural stimulation energy at a second neural stimulation location. The predetermined amount of time can be between about 1 second and about 60 seconds. For example, the predetermined amount of time can be about 5 seconds, about 15 seconds, about 30 seconds, etc.

At block 508, the process 500 can comprise obtaining EMG data characterizing an EMG response during and/or after stimulating each neural stimulation location. As described herein, obtaining the EMG data can comprise detecting an electrical signal (e.g., the EMG response) in the pharynx via a conductive element. The conductive element can be similar to any of the conductive elements disclosed herein. In some embodiments, the conductive element is the same conductive element that was used to apply electrical stimulation energy to the pharynx. In some embodiments, the EMG response is obtained at the potential treatment location at which electrical stimulation energy was previously applied. Additionally or alternatively, the EMG response can be obtained from one or more locations in the pharynx corresponding to muscles of interest for evaluating swallowing function. Obtaining the EMG data can occur simultaneously with applying the neural stimulation energy to one of the neural stimulation sites. Additionally or alternatively, obtaining the EMG data can occur before and/or after applying the neural stimulation energy to one of the neural stimulation sites. Obtaining the EMG data can comprise down-sampling, filtering, amplifying, transforming, and/or otherwise modifying the EMG data to facilitate evaluation, transfer, storage, etc. of the EMG data.

In some embodiments, obtaining the EMG data comprises processing and/or evaluating the EMG data. For example, the EMG data can be decomposed into an MEP amplitude and/or an MEP latency. Additionally or alternatively, a topographical map depicting the cortical representation can be obtained in which an MEP parameter obtained from the EMG data is plotted on a spatial grid of the neural stimulation locations. Other measures of cortical representation can also be obtained from the EMG data such as, but not limited to, a percentage of the neural stimulation locations that, when stimulated, produce an MEP amplitude above a predetermined threshold, a volume of the topographical map, etc.

The particular processes described herein are exemplary only and may be modified as appropriate to achieve the desired outcome. In various embodiments, other suitable methods or techniques can be used to identify a treatment location. Moreover, although various aspects of the methods disclosed herein refer to sequences of steps, in various embodiments the steps can be performed in different orders, two or more steps can be combined together, certain steps may be omitted, and additional steps not expressly discussed can be included in the process as desired.

At least some of the processes described herein can be performed on one computing device or a cluster of computing devices working in concert, or various processes can be performed by remote or distributed computing devices, with different steps being performed by different entities and/or different computing devices. For example, some or all of the processes described herein can be performed in a distributed computing environment in which tasks or modules are performed by remote processing devices, which are linked through a communication network (e.g., a wireless communication network, a wired communication network, a cellular communication network, the Internet, a short-range radio network (e.g., via Bluetooth)). In various embodiments, some or all of the processes described herein can be performed automatically. According to some embodiments, some of the processes described herein may rely at least in part on one or more inputs from a human operator such as a clinician or technician.

### CONCLUSION

Although many of the embodiments are described above with respect to systems, devices, and methods for electrical stimulation of the pharynx, the technology is applicable to other applications and/or other approaches, such as electrical stimulation of the esophagus or trachea. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-5.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method comprising:
via a nasal or oral cavity, positioning an elongate member carrying a conductive element in a lumen of a pharynx of a human subject such that the conductive element is positioned at a first location;
applying electrical stimulation energy at the first location via the conductive element;
after applying the electrical stimulation energy at the first location, obtaining first data characterizing a cortical excitability of the subject;
positioning the elongate member in the lumen such that the conductive element is positioned at a second location;
applying electrical stimulation energy at the second location via the conductive element;
after applying the electrical stimulation energy at the second location, obtaining second data characterizing the cortical excitability of the subject; and
comparing the cortical excitability of the first data to the cortical excitability of the second data.

2. The method of claim 1, further comprising, based on the comparison,
determining whether the application of electrical stimulation energy at the first location or the second location results in greater cortical excitability.

3. The method of claim 1, wherein obtaining at least one of the first data characterizing a cortical excitability or the second data characterizing the cortical excitability includes:
stimulating the subject's neural tissue via transcranial magnetic stimulation (TMS), and
detecting a pharyngeal electromyographic (EMG) response to the stimulation.

4. The method of claim 3, wherein stimulating the subject's neural tissue comprises stimulating one or more regions of the subject's swallow motor cortex.

5. The method of claim 4, wherein stimulating the subject's neural tissue comprises stimulating one or more of the subject's caudal sensory motor cortex, anterior insula, premotor cortex, frontal operculum, anterior cingulate cortex, prefrontal cortex, anterolateral parietal cortex, posterior parietal cortex, precuneus, superiomedial temporal cortex, posterior cingulate cortex, putamen, or caudal nuclei.

6. The method of claim 3, wherein detecting the pharyngeal EMG response includes obtaining a motor-evoked potential (MEP) parameter.

7. The method of claim 6, wherein the MEP parameter includes an MEP amplitude and/or an MEP latency.

8. The method of claim 1, further comprising:
obtaining first baseline data characterizing a cortical excitability of the subject before applying the electrical stimulation energy at the first location;
obtaining second baseline data characterizing a cortical excitability of the subject before applying the electrical stimulation energy at the second location;
comparing the cortical excitability associated with the first data to the cortical excitability associated with the first baseline data; and
comparing the cortical excitability associated with the second data to the cortical excitability of the second baseline data.

9. A method comprising:
obtaining baseline data characterizing a swallowing function parameter of a subject;
delivering electrical stimulation energy at a first location within a pharynx of the subject;
during and/or after delivering energy at the first location, obtaining first data characterizing the swallowing function parameter;
delivering electrical stimulation energy at a second location within the pharynx;
during and/or after delivering energy at the second location, obtaining second data characterizing the swallowing function parameter; and
based on at least two of the baseline data, the first data, or the second data, identifying which of the first or second locations is a preferred treatment location for delivering electrical stimulation energy.

10. The method of claim 9, wherein the swallowing function parameter is a cortical representation associated with the pharynx.

11. The method of claim 9, wherein the swallowing function parameter is a cortical excitability.

12. The method of claim 9, wherein the swallowing function parameter is an outcome measure.

13. The method of claim 9, wherein obtaining the baseline data comprises obtaining first baseline data, the method further comprising, after delivering electrical stimulation energy at the first location and before delivering electrical stimulation energy at the second location, obtaining second baseline data characterizing the swallowing function parameter.

14. The method of claim 9, wherein obtaining the baseline data, the first data, and/or the second data comprises:
applying magnetic stimulation energy to one or more regions of the patient's neural tissue; and
during and/or after applying the magnetic stimulation energy to each of the one or more regions, detecting a pharyngeal EMG response to the magnetic stimulation energy.

15. The method of claim 9, wherein obtaining the baseline data, the first data, and/or the second data comprises performing functional magnetic resonance imaging of the subject's brain.

16. The method of claim 9, wherein identifying which of the first or second locations is the preferred treatment location comprises:
determining a first difference between the baseline data and the first data;
determining a second difference between the baseline data and the second data; and
comparing the first and second differences.

17. The method of claim 16, wherein identifying which of the first or second locations is the preferred treatment location comprises identifying which of the first and second differences is greater.

18. The method of claim 9, wherein identifying which of the first or second locations is the preferred treatment location comprises identifying a percentage of the one or more regions of the patient's neural tissue that, when stimulated, produce a pharyngeal EMG response above a predetermined threshold for the first data and the second data

19. The method of claim 18, wherein identifying which of the first or second locations is the preferred treatment location comprises identifying which of the first or second data has a greater percentage of the one or more regions of the patient's neural tissue that, when stimulated, produce a pharyngeal EMG response above the predetermined threshold.
